(19) United States
(12) Patent Application Publication
(10) Pub. No.: US 2025/0195104 A1
(43) Pub. Date: Jun. 19, 2025

(54) NEEDLE AND CANNULA ASSEMBLY FOR CANNULATION AND TREATMENT OF SUBCUTANEOUS VESSELS

(71) Applicant: Mukesh Gupta, Owensboro, KY (US)

(72) Inventor: Mukesh Gupta, Owensboro, KY (US)

(21) Appl. No.: 19/072,697

(22) Filed: Mar. 6, 2025

Related U.S. Application Data
(63) Continuation of application No. 17/527,538, filed on Nov. 16, 2021.

(51) Int. Cl. A61B 17/34; A61B 17/00

(52) U.S. Cl. CPC A61B 17/3417; A61B 17/3462; A61B 17/3496; A61B 2017/00026; A61B 2017/00778; A61B 2017/00893; A61B 2017/3413; A61B 2017/3419

(57) ABSTRACT

A cannula assembly for treating a puncture site in a subcutaneous vessel includes a cannula and an elongated sleeve with a bore for receiving the cannula. The elongated sleeve includes a plurality of channels circumferentially spaced about the bore through which at least one of a flow of air can be drawn and a plurality of wires can be advanced to promote the formation of a seal between the elongated sleeve and the subcutaneous vessel around the puncture site. The cannula assembly can further include a plug which can be inserted into the bore of the elongated sleeve following removal of the cannula. A needle for insertion into the subcutaneous vessel includes a rigid shaft with an enlarged segment connected to a hub. The needle and cannula assembly can be utilized in a method for cannulating the subcutaneous vessel.

### RELATED APPLICATION

This application is a continuation of U.S. application Ser. No. 17/527,538, filed Nov. 16, 2021, which is incorporated herein by reference in its entirety.

### TECHNICAL FIELD

The present invention relates to assemblies and methods for use in various procedures in which cannulation of a subcutaneous vessel is needed. More particularly, the present invention relates to a needle that exhibits different reflectance characteristics for improved ultrasound visualization, and a cannula assembly that can be used to treat a puncture site within a wall of a subcutaneous vessel and/or to monitor for the development of a hematoma following cannulation.

### BACKGROUND

Many medical procedures require cannulation of subcutaneous vessels, especially arteries. For example, it may be necessary to perform an arteriogram of a leg or heart artery to determine if blockages are present. Cannulation procedures of this type generally require that a percutaneous tubular cannula be inserted through a patient's skin and into a subcutaneous vessel. This of course necessitates that an incision or hole be made in the defining wall of the subcutaneous vessel in order to accommodate the cannula body. Various methods exist for accomplishing this task. For example, FIGS. 1A-IE, in sequence, depict one known cannulation procedure in which a cannula 10 is introduced into a subcutaneous vessel 14 within the body of a patient. As shown, the cannula 10 is of known construction and includes a hollow shaft 12 with a distal end configured to be inserted into the subcutaneous vessel 14 of the patent and a side arm (depicted as a tube), for use in cannulation or catheterization procedures where a passageway is created to a subcutaneous vessel 14, such as a blood vessel, a vein, or an artery. In the known cannulation procedure, an incision is typically made through a skin layer 20 of the body of the patient adjacent to the subcutaneous vessel 14, and the skin layer 20 and the subcutaneous tissue 21 are spread apart by forceps (not shown). A hollow needle 16 with a tip 18 is then inserted into the patient's body through the incision and guided via ultrasonography using an ultrasound device 31 placed on the surface of the skin 20 of the patient until the tip 18 of the needle 16 punctures a wall 22 of the subcutaneous vessel 14, creating a hole 22a (or puncture site 22a) through the wall 22 of the subcutaneous vessel 14, as shown in FIG. 1A. Proper placement of needle 16 can then be confirmed by passage of blood coming out of a proximal hub 24 of the needle 16.

Following insertion of the needle 16, a guidewire 28 is inserted through needle 16 and deposited within the subcutaneous vessel 14, as shown in FIG. 1B. Once the guidewire 28 is deposited, the needle 16 is subsequently removed, as shown in FIG. 1C. The cannula 10, which, in this case, is equipped with a hollow internal dilator 30 is inserted over the guidewire 28 until the distal end of shaft 12 is deposited within the subcutaneous vessel 14, as shown in FIG. 1D. Once the distal end of the shaft 12 of the cannula 10 is deposited within the subcutaneous vessel 14, the guidewire 28 and internal dilator 30 are removed, leaving only the cannula 10, as shown in FIG. 1E. At this point, the cannula 10 is ready for use in the intended medical procedure.

After the medical procedure for which the cannula 10 was inserted is finished, the cannula 10 is removed from the subcutaneous vessel, leaving a puncture site 22a in the subcutaneous vessel 14 which must be closed, especially if the subcutaneous vessel is an artery since the pressure of blood inside an artery is much higher than that of a vein, thereby increasing the bleeding risk from the puncture site in the artery as compared to a vein. Many techniques are thus known for closing a puncture site. The simplest technique to close the puncture site is to apply manual pressure (e.g., by two fingers 33) to the skin overlying the puncture site, as shown in FIG. 1F. Such pressure must be strong enough to be transmitted through the subcutaneous tissue 21 underlying the skin 20 and onto the puncture site 22a in the subcutaneous vessel 14 so that the tissue overlying the puncture site prevents the flow of blood out of the puncture site 22a, thereby leading to formation of blood clot 35 in the puncture site 22a, as also shown in FIG. 1F. Gradually, the blood clot becomes strong enough to keep the puncture site 22a closed, even after manual pressure over the skin is removed. The skin 20 and underlying tissue 21 surrounding the skin puncture site are periodically examined after the application of manual pressure to ensure the blood clot 35 has remained intact in the puncture site 22a in the subcutaneous vessel 14. This is done by visual inspection and palpation. If the puncture site 22a in the subcutaneous vessel 14 is not completely closed, then bleeding occurs into the subcutaneous tissue 21 overlying the subcutaneous vessel 21, thus forming a hematoma 34, palpated as a mass and visualized as a swelling, as shown in FIG. 1G. Such bleeding can also extend out of the skin incision.

The method of insertion of cannula into a subcutaneous vessel and removal of the cannula from a subcutaneous vessel can thus generally be divided into three steps. First, the needle is inserted into the subcutaneous vessel, commonly with ultrasound guidance, followed by insertion of cannula in the manner described above. Second, the cannula is removed and the puncture site within the subcutaneous vessel is closed by manual application of pressure to the skin overlying the hold. Finally, the puncture site is monitored by intermittently examining the puncture site and the skin around it by visual inspection and palpation in the manner described above. However, there are inherent problems with each of these steps.

With respect to visualization of the needle tip using ultrasound guidance, the needle tip and remainder of the shaft of the needle have the same ultrasonic reflectance characteristics, which can mislead a physician to direct the needle tip to the side of a subcutaneous vessel instead of toward the subcutaneous vessel as the needle is advanced. In this regard, the inherent problem of visualizing the tip of the needle is that the tip of the needle and the shaft of the needle are located in a plane different from the plane of the ultrasound waves, thus making it extremely difficult to be certain that the echogenic structure, seen by ultrasound is truly the tip of the needle and not simply a segment of the shaft of the needle. Although attempts have been made in the art to solve this problem (as evidenced, e.g., by U.S. Pat. Nos. 4,401,124, 5,766,135, and 10,188,467), such attempts have not improved the visualization of the tips of needles which are commonly used. Hence, there is a real and unsatisfied need for improving the visualization of the tip of the needle.

With respect to the application of manual pressure of the skin overlying the puncture site in the subcutaneous vessel, such pressure can be quite painful. This is especially true when the subcutaneous vessel is deeper and much larger pressure needs to be applied so that enough pressure can be transmitted through flexible subcutaneous tissue. Additionally, in deeper located subcutaneous vessels, the pressure may not necessarily be transmitted in the intended direction and thus may be less effective in closing the puncture site in the subcutaneous vessel. The inherent problems with manual application of pressure thus include discomfort experienced by the patient during the application of pressure and less than desired success in closing the puncture site in the subcutaneous vessel following removal of the cannula. With respect to the former, discomfort is especially noticeable in obese patients who have subcutaneous vessels that are located more deeply beneath the skin. In such patients, the puncture site in the subcutaneous vessel requires a much larger pressure after removal of the cannula due to the larger depth of intermediary subcutaneous tissue and due to the fact that pressure dissipates as depth increases. Additionally, the area of skin on which pressure is applied is proximal to the site of entry into the skin. As such, the area of skin, on which pressure is applied is at best an estimate and may not necessarily correspond to the vertical position over the puncture site in the deeply located subcutaneous vessel. In such cases, blood may thus continue to exit the puncture site resulting in the formation of hematoma in the subcutaneous tissue or bleeding out of the skin incision.

Finally, with respect to monitoring the puncture site for bleeding and surrounding skin for formation of hematoma by examining intermittently, bleeding may occur and a hematoma may develop in the subcutaneous tissue overlying the subcutaneous vessel puncture site during the periods between examination. If such bleeding is severe, it could be fatal or require transfusion of blood to patient. The inherent problem with monitoring the skin incision for bleeding and surrounding skin for formation of hematoma is thus due to the lack of continuous monitoring. However, no automated means that would provide desired continuous monitoring currently exist in common use.

Accordingly, percutaneous needle and cannula assemblies helping to cure the above-identified problems would be both highly desirable and beneficial.

### SUMMARY

The present invention relates to assemblies and methods for use in various procedures in which cannulation of a subcutaneous vessel is needed. More particularly, the present invention relates to a needle which exhibits different reflectance characteristics for improved ultrasound visualization, and a cannula assembly which can be used to treat a puncture site within a wall of a subcutaneous vessel and/or monitor for the development hematoma following cannulation.

An exemplary cannula assembly for treating a puncture site in a subcutaneous vessel of a patient includes a cannula and an elongated sleeve configured to receive the cannula. The cannula includes a shaft which defines a distal end configured to be inserted into the subcutaneous vessel of the patient. The elongated sleeve includes: a proximal end, a distal end that is configured to be inserted into subcutaneous tissue of the patient and proximal to a puncture site of the subcutaneous vessel, and a bore that extends from the proximal end to the distal end of the elongated sleeve and in which the cannula is received. To promote the formation of a seal between the distal end of the sleeve and the subcutaneous puncture site around the puncture site, the elongated sleeve further includes a plurality of channels circumferentially spaced about the bore through which a flow of air can be drawn and/or a plurality of wires can be advanced into the subcutaneous vessel.

In some embodiments, the proximal end of the elongated sleeve includes a valve which is configured to form a seal around the cannula when the shaft of the cannula is inserted within the bore of the elongated sleeve. In some embodiments, the sleeve is constructed so that the respective channels are interconnected (i.e., in fluid communication with) one another, such that when a port of the sleeve is placed in fluid communication with a vacuum source, a flow of air is drawn upwardly through the respective channels of the plurality of channels.

The cannula assembly can, in some embodiments, further comprises a plug which can be inserted into the bore of the elongated sleeve following removal of the cannula from the sleeve. The length of the plug is such that, in use, the distal end of the plug applies direct pressure to the puncture site when fully inserted into the bore of the elongated sleeve. In some embodiments, a thrombotic material configured to promote thrombosis at the puncture site is deposited on the distal end of the plug. In some embodiments, at least one electrically conductive plate configured to be placed in contact with the puncture site is connected to the distal end of the plug. In such embodiments, the electrically conductive plate(s) is operably connected to an electrical source, such that an electrical current can be selectively applied to the electrically conductive plate(s) to cauterize subcutaneous tissue around the puncture site.

In some embodiments, the elongated sleeve includes a slide mounted for movement with respect to a proximal end of a main body of the elongated sleeve. In such embodiments, the slide is connected to a plurality of wires. In use, the slide can be selectively moved in a first direction to advance the plurality of wires into the subcutaneous vessel to promote the formation of a seal between the distal end of the sleeve and the subcutaneous vessel by urging a wall of the subcutaneous vessel toward the distal end of the sleeve.

In some embodiments, the sleeve of the cannula assembly includes an electrode (or first electrode) that is disposed on an outer surface of the distal end of the elongated sleeve and is configured to transmit electrical current to another electrode (or second electrode) disposed on an exterior surface of the skin of a patient or receive electrical current from the second electrode. In such embodiments, the first electrode and the second electrode are operably connected to a bioimpedance meter configured to measure impedance between the first electrode and the second electrode.

An exemplary needle for insertion into a subcutaneous vessel made in accordance with the present invention includes a rigid shaft and a hub connected to a proximal end of the rigid shaft. The rigid shaft includes a main body that defines a bore for receiving a guide wire that extends from a proximal end to a distal end of the rigid shaft, which terminates in a tip that is configured to puncture the subcutaneous vessel of the patient. The rigid shaft also includes a segment which is enlarged relative to the main body of the rigid shaft. In some embodiments, the enlarged segment may be rounded.

An exemplary method for cannulating a subcutaneous vessel of a patient which utilizes the exemplary needle and cannula assembly of the present invention is also described herein.

### BRIEF DESCRIPTION OF THE DRAWINGS

FIG. 1A is a partial sectional view illustrating a first step in a prior art method for cannulating a subcutaneous vessel;

FIG. 1B is a partial sectional view similar to that of FIG. 1A, but illustrating a second step in the prior art method for cannulating a subcutaneous vessel;

FIG. 1C is a partial sectional view similar to that of FIG. 1B, but illustrating a third step in the prior art method for cannulating a subcutaneous vessel;

FIG. 1D is a partial sectional view similar to that of FIG. 1C, but illustrating a fourth step of the prior art method for cannulating a subcutaneous vessel;

FIG. 1E is a partial sectional view similar to that of FIG. 1D, but illustrating a fifth step of the prior art method for cannulating a subcutaneous vessel;

FIG. 1F is a partial sectional view illustrating a prior art method for promoting the formation of a blood clot following cannulation of a subcutaneous vessel;

FIG. 1G is a partial sectional view similar to that of FIG. 1F, but illustrating dislodging of the blood clot of FIG. 1F and the formation of a hematoma;

FIG. 2A is a partial sectional view illustrating a first step of an exemplary method for cannulating a subcutaneous vessel of a patient in which an exemplary needle made in accordance with the present invention is visualized by ultrasound and inserted through skin, through subcutaneous tissue, and into the subcutaneous vessel of the patient;

FIG. 2B is a partial sectional view similar to FIG. 2A, but illustrating a second step in the exemplary method for cannulating a subcutaneous vessel of a patient in which a guidewire is passed through the exemplary needle of FIG. 2A;

FIG. 2C is a partial sectional view similar to FIG. 2B, but illustrating a third step in the exemplary method for cannulating a subcutaneous vessel of a patient in which the exemplary needle of FIG. 2A is removed, leaving the guidewire of FIG. 2B in place;

FIG. 2D is a partial sectional view similar to FIG. 2C, but illustrating a fourth step in the exemplary method for cannulating a subcutaneous vessel of a patient in which an exemplary cannula assembly made in accordance with the present invention is placed over the guidewire and into place within the subcutaneous vessel;

FIG. 2E is a partial sectional view similar to FIG. 2D, but illustrating a fifth step in the exemplary method for cannulating a subcutaneous vessel of a patient in which the guidewire is removed, leaving the exemplary cannula assembly of FIG. 2D in place;

FIG. 2F is a partial sectional view similar to FIG. 2E, but illustrating a sixth step in the exemplary method for cannulating a subcutaneous vessel of a patient in which a cannula of the exemplary cannula assembly of FIG. 2D is removed, leaving a sleeve of the exemplary cannula assembly in place;

FIG. 2G is a partial sectional view similar to FIG. 2F, but illustrating a seventh step in the exemplary method for cannulating a subcutaneous vessel of a patient in which a plug is inserted through a bore of the sleeve of the exemplary cannula assembly;

FIG. 3 is a side view of the exemplary needle of FIG. 2A;

FIG. 3A is an enlarged partial view of the exemplary needle of FIG. 3;

FIG. 3B is a sectional view of the exemplary needle taken along 3B-3B in FIG. 3A;

FIG. 3C is a sectional view of the exemplary needle taken along 3C-3C in FIG. 3C;

FIG. 4 is a sectional view of the exemplary cannula assembly of FIG. 2E;

FIG. 5 is a sectional view of the sleeve of FIG. 2F;

FIG. 6A is sectional view of the sleeve of FIG. 5 in isolation;

FIG. 6B is a sectional view of the sleeve of FIG. 6A taken along line 6B-6B in FIG. 6A;

FIG. 6C is a partial perspective view of the sleeve of FIG. 6A;

FIG. 6D is a side view of two electrodes for depositing on the skin of the patient and which may be utilized in combination with two electrodes provided on an outer surface of the distal end of the sleeve;

FIG. 6E is a sectional view of the plug of FIG. 2G in isolation;

FIG. 6F is a sectional view of another plug, which may be utilized in the exemplary cannula assembly of FIG. 2D;

FIG. 6G is a cross section view of another plug, which may be utilized in the exemplary cannula assembly of FIG. 2D;

FIG. 6H is a side view an electrically conductive plate deposited on the skin of the patient and which may be utilized in combination with the electrically conductive plate of the plug of FIG. 6G;

FIG. 7 is a sectional view of the exemplary cannula assembly of FIG. 2G;

FIG. 8 is a sectional view of another sleeve for use in the exemplary cannula assembly;

FIG. 9 is another sectional view of the sleeve of FIG. 8;

FIG. 10 is another sectional view of the sleeve of FIG. 8, but with the sleeve of the exemplary cannula assembly placed in the subcutaneous vessel of the patient; and

FIG. 11 is an exploded view of the sleeve of FIG. 8.

### DESCRIPTION OF EXEMPLARY EMBODIMENTS

The present invention includes assemblies and methods for use in various procedures in which cannulation of a subcutaneous vessel is needed. More particularly, the present invention includes a needle that exhibits different reflectance characteristics for improved ultrasound visualization, and a cannula assembly that can be used to treat a puncture site within a wall of a subcutaneous vessel and/or to monitor for the development hematoma following cannulation.

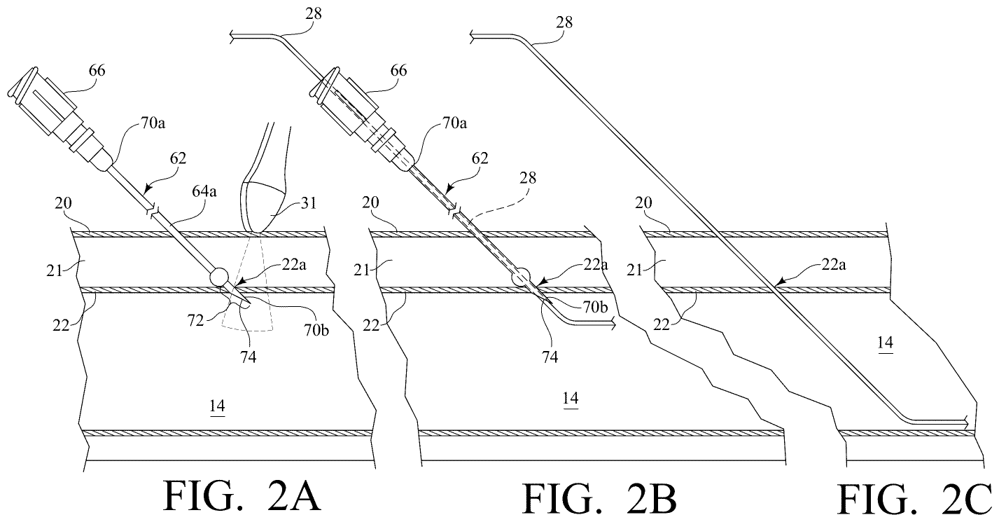

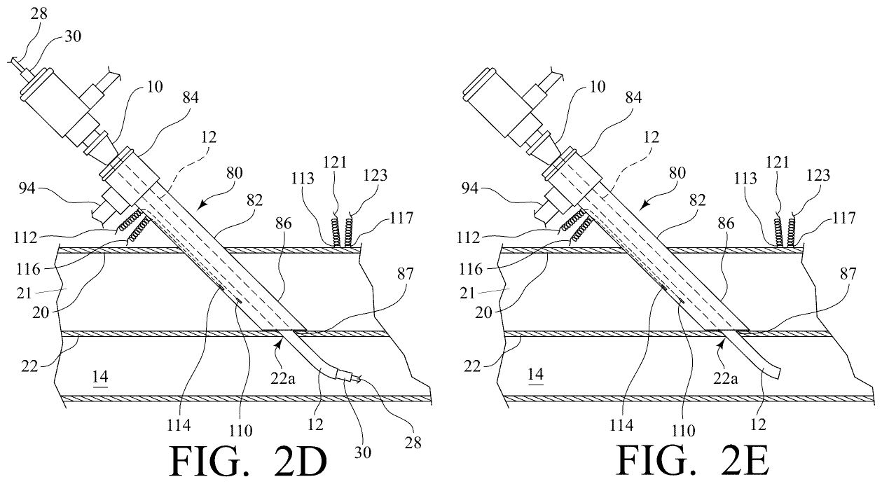

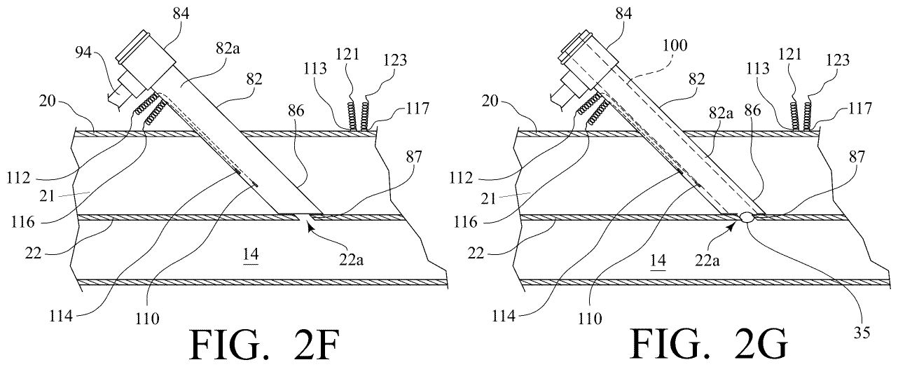

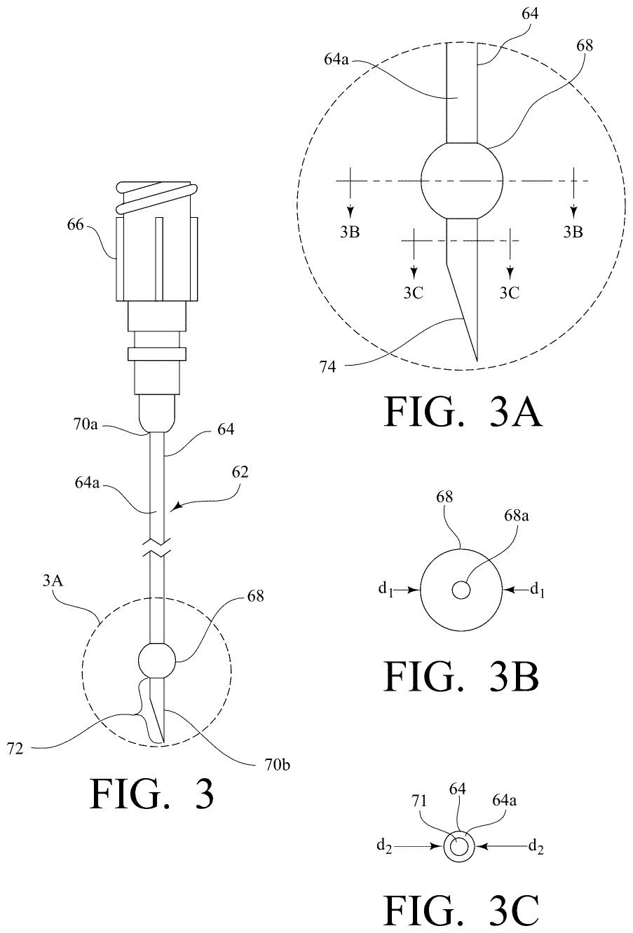

FIGS. 2A-2G, in sequence, show an exemplary method for cannulating a subcutaneous vessel 14 of a patient, which utilizes an exemplary needle 62 (FIGS. 2A and 2B) and an exemplary cannula assembly 80 (2D-2G) made in accordance with the present invention. FIGS. 3 and 3A-C show various views of the exemplary needle 62 or portions thereof. The needle 62 generally comprises a rigid shaft 64 and a hub 66 that is connected to a proximal end of the rigid shaft 64. The rigid shaft 64 defines, and thus can be characterized as including, a main body 64a that defines a bore 71 which extends from the proximal end 70a of the rigid shaft 64 to a distal end 70b of the rigid shaft 64. The distal end 70b of the rigid shaft 64 terminates in a tip 74 to facilitate puncturing of the wall 22 of the subcutaneous vessel 14, as further described below. In this exemplary embodiment, the bore 71 of the rigid shaft 64 is of sufficient diameter to receive a guidewire 28. The hub 66 of the needle 62 defines a lumen, which feeds into the bore 71 of the rigid shaft 64, such that the lumen of the hub 66 and the bore 71 of the rigid shaft 64 define a pathway through which the guidewire 28 can travel through the needle 62 for deposit into the subcutaneous vessel 14. Unlike needles of known construction, the rigid shaft 64 of the needle 62 further includes a segment 68 (or enlarged segment 68), which is enlarged relative to the remainder of the main body 64a of the rigid shaft 64, and is positioned a predetermined distance 72 from the tip 74 of the rigid shaft 64.

Referring now more particularly to FIGS. 2A, 2B, 3, and 3A-3C, in this exemplary embodiment, the enlarged segment 68 is rounded and generally has the shape of a sphere with a radius ranging from about 2 mm-4 mm. Of course it is appreciated that the shape and size of the enlarged segment 68 can vary to accommodate different subcutaneous environments and applications without departing from the spirit and scope of the present invention, but the enlarged segment 68 generally exhibits a diameter, d₁, which is greater than the diameter, d₂, than the distal end 70b of the rigid shaft 64, as shown in FIGS. 3B and 3C. Further, it should be appreciated that the predetermined distance between the tip 74 of the rigid shaft 64 and the enlarged segment 68 may also vary depending on the known thickness of the wall 22 of the subcutaneous vessel 14 intended for cannulation. For instance, the enlarged segment 68 may be spaced a predetermined distance of approximately 2.5 mm for implementations in which the subcutaneous vessel 14 is a femoral artery, while the enlarged segment 68 may be spaced a predetermined distance of approximately 2 mm for implementations in which the subcutaneous vessel 14 is a radial artery.

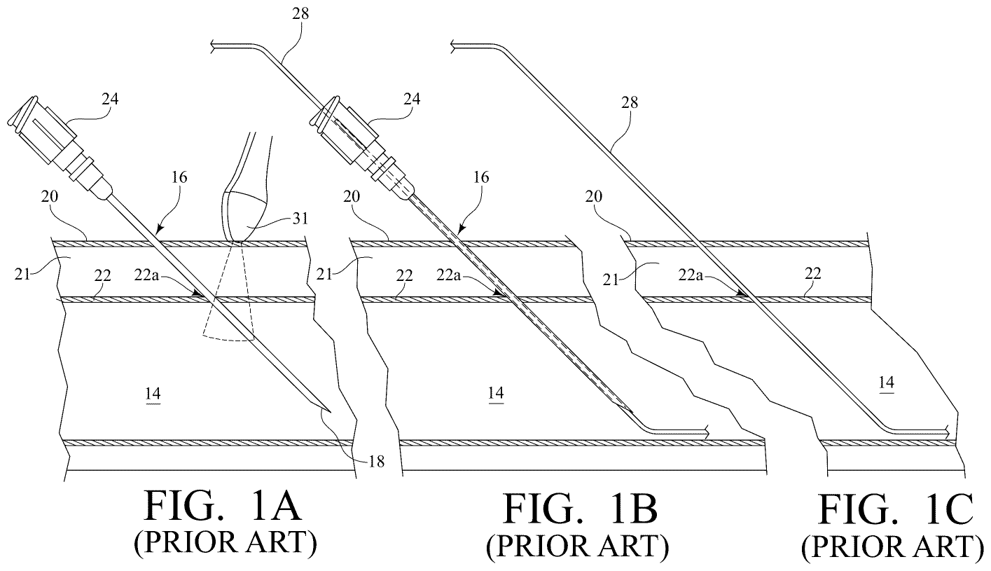

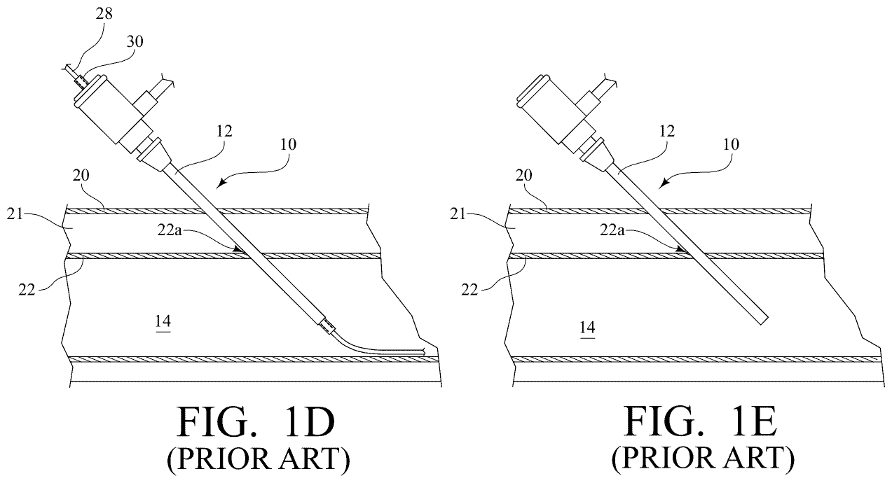

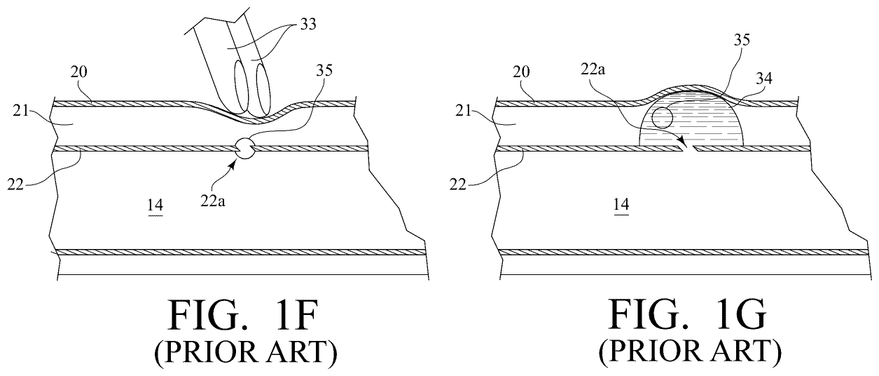

Referring still to FIGS. 2A, 2B, 3, and 3A-3C, the exemplary method for cannulating a subcutaneous vessel 14 of a patient (or exemplary method) commences with the tip 74 of the rigid shaft 64 of the needle 62 being inserted through the wall 22 and into the subcutaneous vessel 14 of the patient. As in the prior art method described above with reference to FIGS. 1A-1E, in this implementation, an ultrasound device 31, or component thereof, such as a wand, is placed on the skin layer 20 overlying the subcutaneous vessel 14, is utilized to help visualize and assist a physician in guiding the needle 62 into the subcutaneous vessel 14 until the tip 74 of the needle 62 punctures the wall 22, thus creating a puncture site 22a through the wall 22 of the subcutaneous vessel 14. Ultrasound devices which may be utilized in the above-described manner are readily known within the art, and, as such, are not described herein.

Referring still to FIGS. 2A, 2B, 3, and 3A-3C, unlike the prior art method described above with reference to FIGS. 1A-1E, the enlarged segment 68 of the rigid shaft 64 of the needle 62, exhibits different ultrasound reflectance characteristics than the main body 64a of the rigid shaft 64. In other words, the enlarged segment 68 will reflect sound waves back to the ultrasound device 31 in a manner that is different from the manner in which the other portions of the main body 64a reflect the sound waves and thus will appear as a different visual profile (echogenic structure) on a display (not shown) of the ultrasound device 31 than the other portions of the main body 64a of the rigid shaft 64. Accordingly, because the enlarged segment 68 is a known, predetermined distance 72 from the tip 74 of the rigid shaft 64, the enlarged segment 68 thus provides an approximation as to where the tip 74 of the rigid shaft 64 is relative to the subcutaneous vessel 14, thereby enabling a physician to more accurately direct the tip 74 into the subcutaneous vessel 14. Furthermore, because the enlarged segment 68 of the rigid shaft 64 is larger than the puncture site 22a created by the distal end 70b of the rigid shaft 64, the enlarged segment 68 also lowers the risk of the needle 62 penetrating beyond the lumen of the subcutaneous vessel 14 by offering resistance.

Referring still to FIGS. 2A, 2B, 3, and 3A-3C, in this exemplary embodiment, the rigid shaft 64 of the needle 62 is constructed of metal (e.g., stainless steel) and the hub 66 is constructed of synthetic resin material. In this exemplary embodiment, the enlarged segment 68 is constructed by welding and then smoothing the surface. However, in alternative embodiments, the enlarged segment 68 may be constructed separately, e.g., as a solid ball, with hollowed lumen 68a through which the main body 64a of the rigid shaft 64 is inserted and adhered to the main body 64a of the rigid shaft 64. In yet another alternative embodiment, the hollowed out lumen 68a of the enlarged segment 68 may include a first set of threading that is configured to interlock with a second set of threading provided on to the distal end 70b of the rigid shaft 64 of the needle 62.

Prior to insertion of the needle 62 into subcutaneous vessel 14, in some implementations, an incision may be made through a layer of skin 20 of the patient's body adjacent to the subcutaneous vessel 14. Following the incision, the layer of skin 20 and the subcutaneous tissue 21 may then be spread apart by forceps (not shown) to facilitate insertion of the needle 62 in the manner described above. In some implementations, proper placement of tip 74 of the rigid shaft 64 may be confirmed by passage of blood out of the hub 66 of the needle.

Referring now to FIGS. 2B and 2C, following insertion of the needle 62, in the next step of the exemplary method, a guidewire 28 (represented in FIG. 2B as a mixture of solid and broken lines and in FIG. 2C with only solid lines) is inserted through the lumen of the hub 66 and the bore 71 of the needle 62 until the distal end of the guidewire 28 is deposited within the subcutaneous vessel 14. Following deposit of the guidewire 28, the needle 62 is then removed from the patient while leaving the guidewire 28 in place, as shown in FIG. 2C. After the needle 62 is removed, the cannula assembly 80 is then inserted into the patient, as further described below.

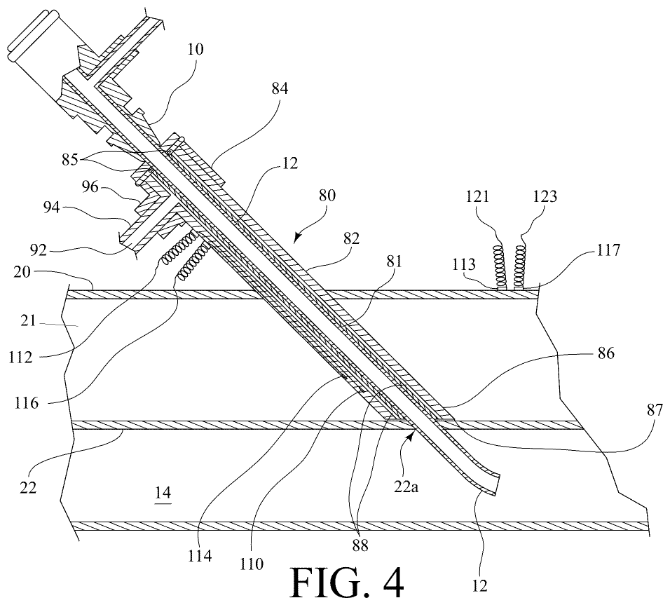

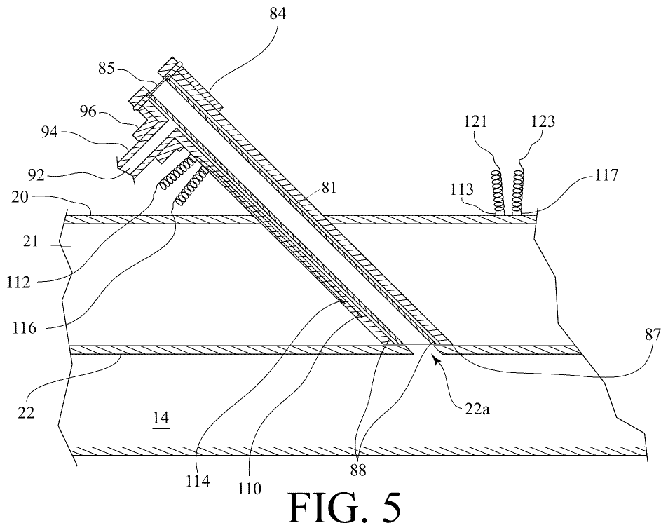

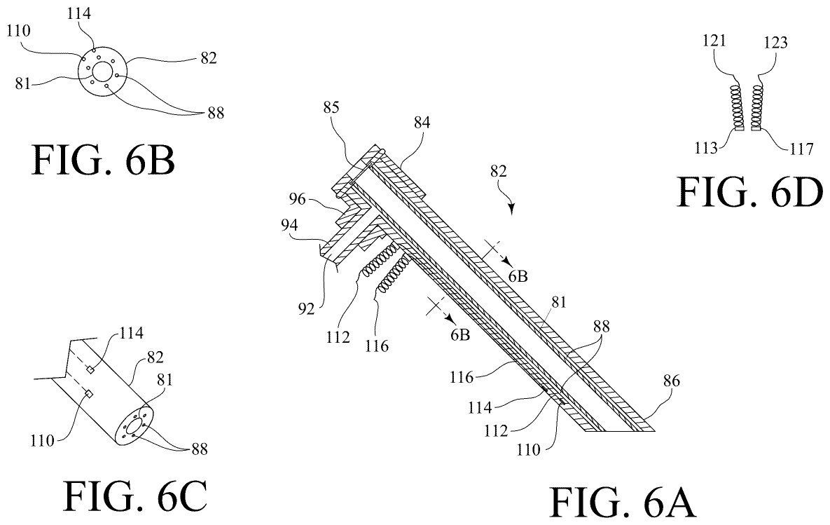

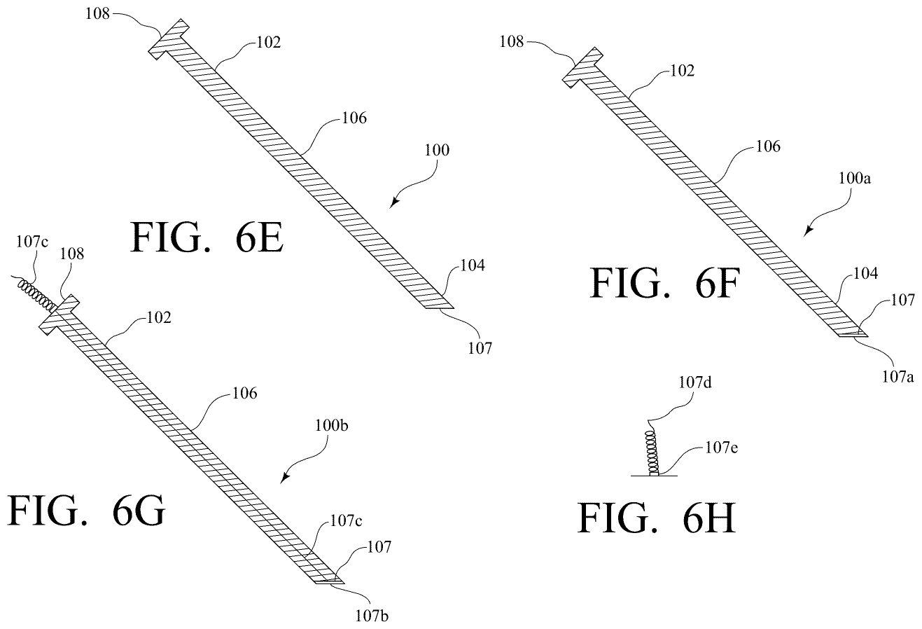

Referring now to FIGS. 2D, 2E, 4, 5, and 6A, FIG. 4 shows a sectional view in which the cannula assembly 80 is inserted within the patient. FIG. 5 shows a sectional view similar to FIG. 4, but with the cannula 10 of the cannula assembly 80 removed. FIGS. 6A-6C then show various views of the sleeve 82 of the cannula assembly 80 or portions thereof. The cannula assembly 80 generally comprises a cannula 10 and an elongated sleeve 82 (or sleeve 82) configured to receive the cannula 10. In this exemplary embodiment, the cannula 10 is of the same construction and provides the same functionality as the cannula 10 described above with reference to FIGS. 1D and 1E, and, as such, is provided with the same reference numeral within the drawings. In this regard, like components and environments are provided with like reference numerals throughout the present application. The sleeve 82 defines, and thus can be characterized as including: a proximal end 84; a distal end 86 configured to be inserted into the subcutaneous tissue 21; and a bore 81 of sufficient dimension to accommodate the shaft 12 of the cannula 10 and extending from the proximal end 84 to the distal end 86 of the sleeve 82. Preferably, the bore 81 of the sleeve 82 has a diameter which corresponds to that of the shaft 12 of the cannula 10, such that, when the cannula 10 and sleeve 82 are combined, the shaft 12 frictionally engages an interior surface of the sleeve 82 so that the sleeve 82 is positioned about, and is supported by, the shaft 12 of the cannula 10. In this exemplary embodiment, the proximal end 84 of the sleeve 82 includes a valve 85, configured to transition between an open position (FIG. 4) when the shaft 12 of the cannula 10 is inserted into the bore 81 and a closed position (FIG. 5) when the shaft 12 of the cannula 10 is removed from the bore 81. The valve is preferably constructed in a manner and of a material such that, when the shaft 12 of the cannula 10 is inserted and the valve 85 is in the open position, the valve 85 forms a seal around the shaft 12 of the cannula 10.

Referring now to FIGS. 4 and 6A-6C, the sleeve 82 further defines, and thus can be further characterized as including, a plurality of channels 88 circumferentially spaced about the bore 81. The sleeve 82 also includes, in this exemplary embodiment, a tubing 94, which defines a port 92 that is in fluid communication with the plurality of channels 88. In this exemplary embodiment, the sleeve 82 is constructed so that the respective channels 88 are interconnected to (i.e., in fluid communication with) one another, such that, when the port 92 is placed in fluid communication with a vacuum source (not shown), a flow of air and/or liquid is drawn upwardly through the respective channels of the plurality of channels 88. In this exemplary embodiment, the plurality of channels 88 are connected to the tubing 94 at the proximal end 84 of the sleeve 82, and the tubing 94 is connected to a main body of the sleeve 82 by a connector 96, which, in this case, defines a port in which the tubing 94 is inserted. Alternative embodiments are, however, contemplated in which the tubing 94 and main body of the sleeve 82 are integrally formed (i.e., of a unitary construction).

Referring now to FIGS. 2D-2G, 4, 5, and 6A, the distal end 86 of the sleeve 82 terminates at a tip 87, which is configured to cup the subcutaneous vessel 14 when placed in contact therewith. To this end, in this exemplary embodiment, the distal end 86 of the sleeve 82 defines a 450 angle. In some embodiments, the edges of the tip 87 which, in use, come into contact with the subcutaneous vessel 14 may also be curved or notched to better follow the contours of the subcutaneous vessel 14.

In this exemplary embodiment, the sleeve 82 of the cannula assembly is constructed of a synthetic plastic material via injection molding or three-dimensional printing.

Referring now even more specifically to FIGS. 2D and 2E, in the exemplary method, following removal of the needle 62 from the patient, the cannula assembly 80 is inserted into the patient so that the distal end of the shaft 12 of the cannula 10 is inserted into the subcutaneous vessel 14 and the distal end 86 of the sleeve 82 is proximally positioned next to the puncture site 22a while the proximal end 84 of the sleeve 82 is positioned outside of the body of the patient. As with insertion of the needle 62, prior to insertion of the cannula assembly 80, in some implementations, an incision within the layer of skin 20 of the patient may be spread apart using forceps (not shown) to facilitate insertion of the cannula assembly 80 in the manner described above. In this implementation, and referring now specifically to FIG. 2D, the distal end of the shaft 12 of the cannula 10 is inserted into the subcutaneous vessel 14 over an internal dilator 30, which, in turn, is provided over the guidewire 28 deposited within the subcutaneous vessel 14. As shown in FIG. 2E, once the distal end of the cannula 10 is deposited within the subcutaneous vessel 14, the internal dilator 30 and guidewire 28 are retracted through the cannula assembly 80 and removed from the patient so that the cannula 10 can be utilized in an intended vascular procedure.

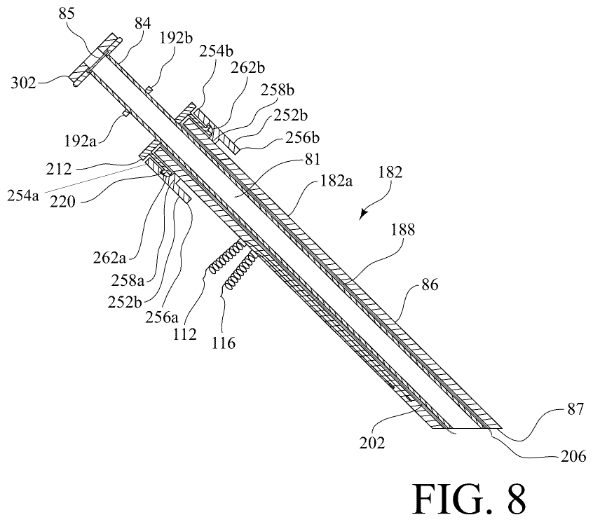

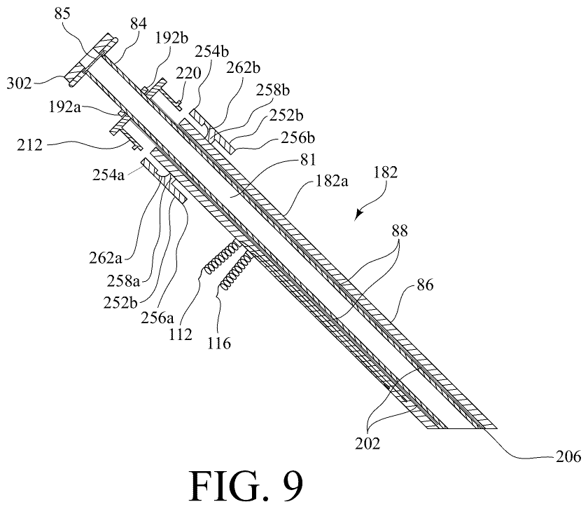

Referring now to FIGS. 2F, 2G, and 6A-6C, after the intended vascular procedure is finished, in the exemplary method, the cannula 10 is retracted through the sleeve 82 and removed from the subcutaneous vessel 14. In this implementation, prior to removal of the cannula 10, the port 92 of the tubing 94 is placed in fluid communication with a vacuum source (not shown), thus causing a flow of air and/or fluid to be drawn through the openings of the plurality of channels 88 and the sleeve 82 to apply a suction force. The suction force applied by the sleeve 82 promotes anchoring of the distal end 86 of the sleeve 82 to the subcutaneous vessel 14 and the formation of a seal between the distal end 86 of the sleeve 82 and the wall 22 of the subcutaneous vessel 14 around the puncture site 22a. Alternative embodiments and implementations are, however, contemplated in which the formation of a seal between the sleeve of the cannula assembly 80 and wall 22 of the subcutaneous vessel 14 is achieved by alternative anchoring means, as further described below with reference to FIGS. 8-11.

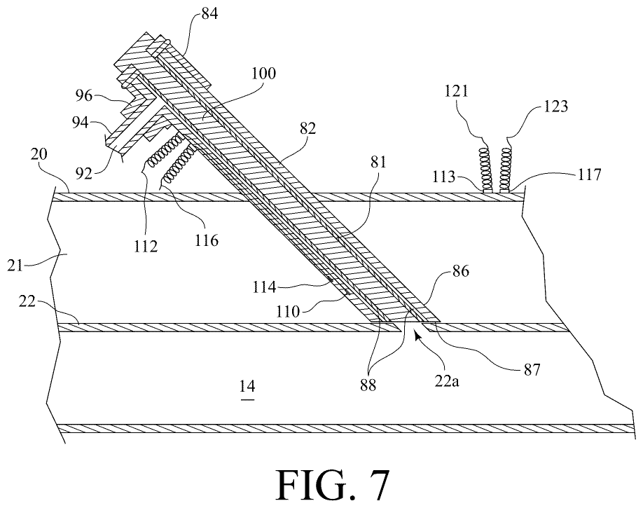

Referring now to FIGS. 2G, 6E, and 7, FIGS. 6E-6G are sectional views of various plugs 100, 100a, 100b which may be utilized with the sleeve 82 following removal of the cannula 10. FIG. 7 then provides a sectional view of the sleeve 82 and plug 100 inserted within a patient. In the exemplary embodiment shown in FIGS. 2G, 6E, and 7, the cannula assembly 80 further includes a plug 100, which is inserted through the proximal end 84 of the sleeve 82 to apply direct pressure around the puncture site 22a following removal of the cannula 10 and to promote the formation of a blood clot 35. As shown, the plug 100 is comprised of an elongated rod 106, with a distal end 104 that terminates in a tip 107 and an enlarged proximal end 102, which, in this case, defines a disc 108 with a diameter greater than that of the opening of the bore 81 at the proximal end 84 of the sleeve 82. The disc 108 of the enlarged proximal end 102 of the plug 100 limits the extent to which the plug 100 can be inserted within the bore 81 of the sleeve 82. The shape of the of the tip 107 of the distal end 104 is preferably configured to follow the contour of the wall 22 of the subcutaneous vessel 14, and thus, in some embodiments, may retain a cup-like shape. The length of the plug 100 is such that, in use, the distal end of the plug 100 applies direct pressure to the puncture site 22a when fully inserted into the bore 81 of the sleeve 82, and the diameter of the plug 100 is such that the plug 100 frictionally engages an interior surface of the sleeve 82 within the bore 81 to hold the plug 100 in position and maintain pressure on the puncture site 22a.

As a result of the disc 108 of the enlarged proximal end 102 of the plug 100 limiting the extent to which the plug 100 can be inserted into the bore 81 of the sleeve 82 and the distal end 86 of the sleeve 82 being positioned as to circumscribe the puncture site 22a, the magnitude of direct pressure applied by the plug 100 to the wall 22 of the subcutaneous vessel 14 is, in turn, limited. The potential magnitude of direct pressure applied by the plug 100 to the wall 22 of the subcutaneous vessel 14 is thus much less than that typically employed using manual pressure (FIG. 1F) to promote formation of a blood clot 35. In this way, the sleeve 82 and plug 100 of the cannula assembly 80 can thus be used to promote the formation of a blood clot 35 while simultaneously limiting patient discomfort. In addition to promoting the formation of a blood clot 35, the pressure applied by the plug 100 also serves to reduces the risk of leakage of blood flow from the puncture site 22a into the subcutaneous tissue 21 above the subcutaneous vessel 14.

FIG. 6F shows another plug 100a which may alternatively be utilized in the cannula assembly 80. As shown, in this exemplary embodiment, the plug 100a includes the same features and functions in the same manner as the plug 100 described above with reference to FIGS. 2G, 6E, and 7. However, in this embodiment, the tip 107 of the plug 100a is coated with thrombin 107a (i.e., thrombin 107a is deposited on the tip 107) to promote thrombosis at the puncture site 22a. Of course, it should be appreciated that other thrombus promoting products (i.e., thrombotic materials), such as fibrinogen, may alternatively be utilized without departing from the spirit or cope of the present invention.

FIG. 6G shows another plug 100b which may alternatively be utilized in the cannula assembly 80. As shown, in this exemplary embodiment, the plug 100a includes the same features and functions in the same manner as the plug 100 described above with reference to FIGS. 2G, 6E, and 7. In this embodiment, however, at least one electrically conductive plate 107b, such as a metal plate, configured to be placed in contact with the puncture site 22a is connected to the distal end 104 of the plug 100b. Specifically, in this embodiment, a single electrically conductive plate 107b is connected to the tip 107 of the plug 100b. The electrically conductive plate 107b is, in turn, operably connected to an electrical source (not shown), such as a cauterization machine of known construction, via an electrically conductive wire 107c that extends from the tip 107, through the body, and out of the distal end 104 of the plug 100b, such that the electrical source can be selectively activated to supply the electrically conductive plate 107b (or electrode) with electrical current to cauterize the tissue of the wall 22 of the subcutaneous vessel 14 surrounding the puncture site 22a. In this implementation an additional (or second) electrically conductive wire 107d is connected to another electrically conductive plate 107e deposited on the skin 20 of the patient and is also operably connected to the electrical source, as shown in FIG. 6H. In this regard, the skin 20 of the patient thus serves as a second electrode, which, in combination with the electrically conductive plate 107b on the tip 107 of the plug 100, effectively forms a circuit through which an electrical current can travel to and from the electrical source. Alternative embodiments are, however, contemplated in which a second electrically conductive plate is connected to the tip 107 of the plug 100b at a predetermined distance, i.e., spaced apart, from the first electrically conductive plate 107b to establish a circuit for the flow of electrical current. In such embodiments, the second electrically conductive plate is operably connected to the electrical source via a second electrically conductive wire that extends from the tip 107, through the body, and out of the distal end 104 of the plug 100b.

Referring now specifically to FIG. 6A, FIG. 6C, and FIG. 7, in this implementation, following treatment of the puncture site 22a in the manner described above, the exemplary method concludes with monitoring of the area around the puncture site 22a for the development of a hematoma. To this end, in this exemplary embodiment, the sleeve 82 of the cannula assembly 80 further includes one or more electrodes 110, 114, which are provided on the outer surface of the distal end 86 of the sleeve 82. Specifically, in this embodiment, the sleeve 82 of the cannula assembly 80 includes two such electrodes: a first electrode 110; and a second electrode 114. In use, the first and second electrodes 110 and 114 are in close proximity to puncture site 22a and are operably connected to a bioimpedance meter (not shown) by a first wire 112 and a second wire 116, respectively, that extend through the main body 82a and out of the proximal end 84 of the sleeve 82.

Referring now to FIGS. 6A-6D and 7, the one or more electrodes 110, 114 provided on the outer surface of the distal end 86 of the sleeve 82 work in conjunction with one or more corresponding electrodes 113, 117 attached to a portion of the skin 20 proximal to the puncture site 22a of the subcutaneous vessel 14. For example, in instances where cannulation was performed on the femoral artery, the one or more electrodes 113, 117 attached to skin 20 located on the same side of the anterior pelvis, or, alternatively, the same side hip area. In this implementation, there are two electrodes attached to the skin 20 of the patient: a third electrode 113 corresponding to the second electrode 114 provided on the distal end of the sleeve 82; and a fourth electrode 110 corresponding to the first electrode 110 provided on the distal end of the sleeve 82. The third electrode 113 and the fourth electrode 117 attached to the skin 20 of the patient are operably connected to the bioimpedance meter by a third wire 121 and a fourth wire 123, respectively. In use, the first electrode 110 and the third electrode 113 are configured to send a current to the second electrode 114 and the fourth electrode 117, respectively, which is subsequently measured by the bioimpedance meter to provide an impedance reading. Such readings may be taken substantially continuously or at predetermined time intervals programmed into the bioimpedance meter or a controller (e.g. microcontroller including a processor configured to execute instructions stored in a memory component) configured to control operation of the bioimpedance meter while the sleeve 82 is present in the patient.

To detect the development of a hematoma around the puncture site 22a, an initial impedance reading or set of impedance readings may be taken to establish a baseline to which subsequent impedance readings can be compared, with a change in impedance (e.g., either generally or within a predefined threshold) signaling the development of a hematoma. In some implementations, the bioimpedance meter may be operably connected to an alarm system, such that when a change in impedance occurs, the alarm system emits a cue to alert a medical professional of the same so that the patient can be treated accordingly. In this way, the cannulation method of the present invention can thus be used to monitor the development of a hematoma during intervals in which the patient is not under observation by a medical professional.

Although the sleeve 82 is described as both treating the puncture site 22a of the subcutaneous vessel 14 and monitoring the development of a hematoma around the puncture site 22a in the exemplary cannulation method described above, it should be appreciated that the sleeve 82 can alternatively be utilized exclusively as a hematoma monitoring device or as a device to treat the puncture site 22a.

FIGS. 8-11 show an alternative sleeve 182 which may be utilized in the cannula assembly 80, in place of the sleeve 82 described above with reference to FIGS. 2D-2G, 4, 5, 6A-6C, and 7. In this exemplary embodiment, the sleeve 182 generally includes the same features and functions in the same manner as the sleeve 82 described above with reference to FIGS. 2D-2G, 4, 5, 6A-6C, and 7, except that, instead of a suction force, the sleeve 182 is configured to be anchored to the wall 22 of the subcutaneous vessel 14 by a pulling force. In this regard, instead of tubing 94 configured to place the sleeve 182 in fluid communication with a vacuum source, the sleeve 182 includes a slide 212 connected (e.g., by adhesion) to a plurality of wires 202 which are configured to engage the wall 22 of the subcutaneous vessel 14. When the sleeve 182 is assembled, each respective wire of the plurality of wires 202 is inserted, at least partially, in one of the plurality of channels 88, which, in this case, are defined by a middle segment of the main body 182a of the sleeve 182 that is enlarged relative to the proximal end 84 of the main body 182a of the sleeve 182. In this exemplary embodiment, the proximal end 84 of the main body 182a of the sleeve 182 is capped with a disc-shaped member 302, which includes the valve 85. The slide 212 is mounted for movement with respect to the proximal end 84 of the sleeve 182, and, in this exemplary embodiment, comprises a tubular member with a central bore 212a configured to receive a proximal end of the main body 182a of the sleeve 182. Preferably, the central bore 212a has a diameter such that an interior surface of the slide 212 frictionally engages the main body 182a of the sleeve 182 as the slide 212 is moved.

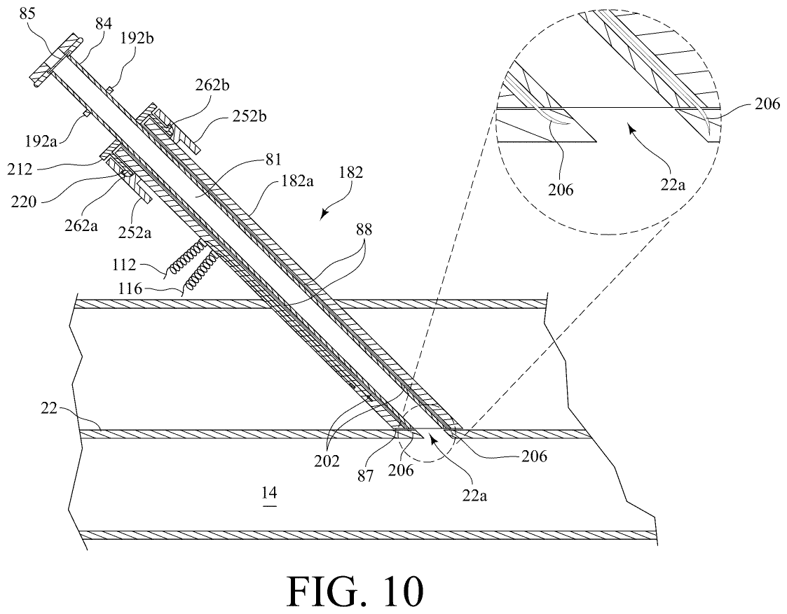

Referring still to FIGS. 8-11, in use, the slide 212 can be selectively moved in a first (downward) direction to advance the plurality of wires 202 attached thereto within the plurality of channels 88, thereby directing a distal end 206 of each respective wire into the wall 22 of the subcutaneous vessel 14, as shown in FIGS. 8 and 10. As shown in FIGS. 8, 10, and 11, each wire of the plurality of wires 202 is preferably comprised of a semi-rigid material, such as stainless steel, which is biased towards a curved configuration at its distal end 206, such that, as the plurality of wires 202 are advanced out of the plurality of channels 88 and beyond the tip 87 of the sleeve 182, the distal end 206 of each respective wire transitions from a straightened to hook-like shape. Accordingly, as the plurality of wires 202 enter the wall 22 of the subcutaneous vessel 14 and transition to the curved configuration, the wall 22 of the subcutaneous vessel 14 is urged toward the sleeve 182 to promote the formation of a seal between the tip 87 of the sleeve 182 and the subcutaneous vessel 14 around the puncture site 22a, thereby permitting treatment of the puncture site 22a in the various manners described herein. Once the distal end 206 of the plurality of wires 202 is deposited within the wall 22 of the subcutaneous vessel 14, the slide 212 can then be moved in a second (upward) direction to retract the plurality of wires 202 back into the plurality of channels 88 to facilitate removal of the sleeve 182 from the patient.

Referring still to FIGS. 8-11, in this exemplary embodiment, movement of the slide 212 in the upward direction is limited by one or more protrusions 192 (or bars) connected to a first (upper) portion of the main body 182a of the sleeve 182, and movement of the slide 212 in the downward direction is limited by one or more clips 252a, 252b connected to a second (middle) portion of the main body 182a of the sleeve 182. Specifically, in this embodiment, the sleeve 182 includes two opposing bars 192 which radially extend from an exterior surface of the main body 182a of the sleeve 182 and two opposing clips 252a, 252b attached to the outer surface of the main body 182a of the sleeve 182. In this exemplary embodiment, each clip is substantially T-shaped, with a proximal end 254a, 254b and an opposing distal end 256a, 256b interconnected by a middle leg 258a, 258b, which serves as the point of attachment to the main body 182a of the sleeve 182. To maintain the plurality of wires 202 in an advanced position when the slide 212 is moved in the downward direction, in this exemplary embodiment, an inner face of the proximal end 254a, 254b of each clip 252a, 252b defines a recess 262a, 262b configured to receive a circumferential ridge 220 of the slide 212. To facilitate insertion of the circumferential ridge 220 of the slide 212 into the recess defined in the proximal end 254a, 254b of each clip 252a, 252b, each clip 252a, 252b is constructed such that the distal end 256a, 256b of each clip can be pressed inwardly (i.e., toward the main body 182a of the sleeve 182) to cause the proximal end 254a, 254b of each clip 252a, 252b to move outwardly (i.e., away from the main body 182a of the sleeve 182) to provide sufficient space for the circumferential ridge 220 to be received. Once received, the pressure on the distal end 256a, 256b of each clip 252a, 252b can then be released to maintain the circumferential ridge 220 within the recess 262a, 262b and prevent the plurality of wires 202 from being inadvertently retracted from the wall 22 of the subcutaneous vessel 14. In this regard, the circumferential ridge 220 and one or more clips 252a, 252b can thus be characterized as defining a locking mechanism.

Although the various plugs 100, 100a, 100b are described herein primarily in the context of being utilized in combination with the sleeve 82 described herein with reference to FIGS. 2D-2G, 4, 5, 6A-6C, and 7, it should be appreciated that the plugs 100, 100a, 100b may be utilized in the same fashion with the sleeve 182 described herein with reference to FIGS. 8-11.

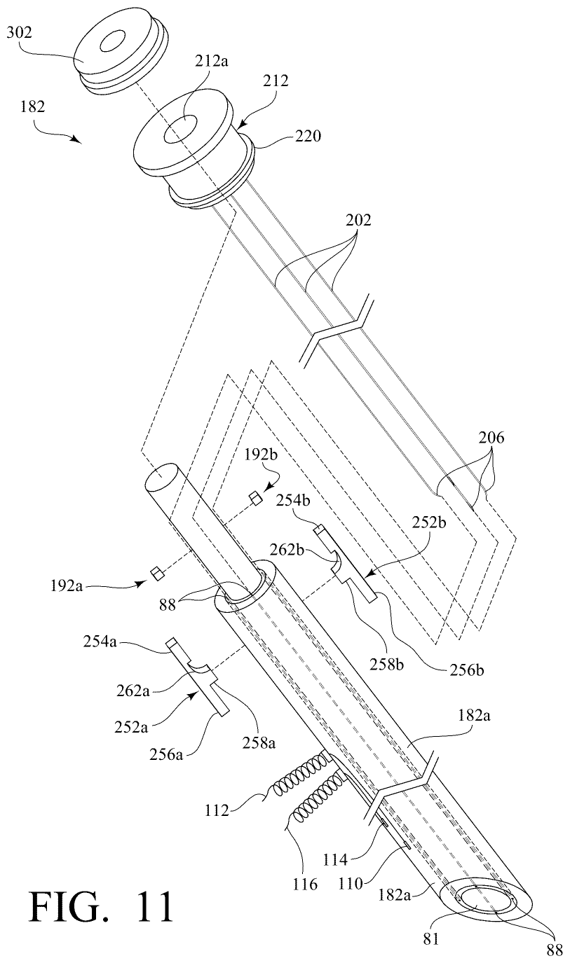

In this exemplary embodiment, the various components of the sleeve 182 are constructed of a synthetic plastic via injection molding or three-dimensional printing, except for the plurality of wires 202, which, as noted above are constructed of a metal, such as stainless steel. As perhaps best shown in FIG. 11, in this exemplary embodiment disc-shaped member 302, protrusions 192, and clips 252a, 252b are heated and fused to the main body 182a of the sleeve 182. Of course, alternative means of connection may alternatively be employed without departing from the spirit or scope of the present invention.

One of ordinary skill in the art will recognize that additional embodiments and implementations are also possible without departing from the teachings of the present invention or the scope of the claims which follow. This detailed description, and particularly the specific details of the exemplary embodiments disclosed herein, is given primarily for clarity of understanding, and no unnecessary limitations are to be understood therefrom, for modifications will become apparent to those skilled in the art upon reading this disclosure and may be made without departing from the spirit or scope of the claimed invention.

What is claimed is:

1. A cannula assembly for treating a puncture site in a subcutaneous vessel of a patient, comprising:
a cannula including a shaft, the shaft defining a distal end of the cannula that is configured to be inserted into the subcutaneous vessel of the patient; and
an elongated sleeve, the elongated sleeve including
a proximal end,
a distal end configured to be inserted into the subcutaneous tissue and positioned proximal to the puncture site of the patient,
a bore extending from the proximal end of the elongated sleeve to the distal end of the elongated sleeve and configured to receive the shaft of the cannula,
a slide mounted for movement with respect to a proximal end of a main body of the elongated sleeve, the slide connected to each wire of a plurality of wires, and
a plurality of channels circumferentially spaced about the bore;
wherein the plurality of channels define a pathway through which at least one of (i) a flow of air can be drawn and (ii) the plurality of wires can be advanced to promote the formation of a seal between the distal end of the elongated sleeve and the subcutaneous vessel around the puncture site, and
wherein, in use, the slide can be selectively moved in a first direction to advance the plurality of wires within the plurality of channels and direct a distal end of the plurality of wires into the subcutaneous vessel of the patient, and wherein the slide can be selectively moved in a second direction to retract the plurality of wires within the plurality of channels.

2. The cannula assembly according to claim 1, wherein the elongated sleeve includes one or more protrusions radially extending from an exterior surface of the elongated sleeve and one or more clips configured to receive a circumferential ridge of the slide, and wherein the one or more clips limit the extent to which the slide can move in the first direction and the one or more protrusions limit the extent to which the slide can move in the second direction.

3. The cannula assembly according to claim 1, wherein the proximal end of the elongated sleeve includes a valve configured to form a seal around the cannula when the shaft of the cannula is inserted within the bore of the elongated sleeve.

4. The cannula assembly according to claim 1, wherein the plurality of channels are interconnected.

5. The cannula assembly according to claim 1, and further comprising a plug configured to be inserted to the bore of the elongated sleeve,
wherein, in use, a distal end of the plug applies a pressure to the puncture site when the plug is fully inserted into the bore of the elongated sleeve.

6. The cannula assembly according to claim 5, wherein a thrombotic material configured to promote thrombosis at the puncture site is deposited on the distal end of the plug.

7. The cannula assembly according to claim 5, wherein at least one electrically conductive plate configured to be placed in contact with the puncture site is connected to the distal end of the plug, and wherein the at least one electrically conductive plate is operably connected to an electrical source.

8. The cannula assembly according to claim 1, and further comprising a first electrode disposed on an outer surface of the distal end of the elongated sleeve, the first electrode being configured to transmit electrical current to a second electrode disposed on an exterior surface of the skin of the patient or receive electrical current from the second electrode,
wherein the first electrode and the second electrode are operably connected to a bioimpedance meter configured to measure impedance between the first electrode and the second electrode.

9. A needle for insertion into a subcutaneous vessel of a patient, comprising:
a rigid shaft, including
a main body defining a bore for receiving a guidewire, the bore extending from a proximal end of the rigid shaft to a distal end of the rigid shaft, the distal end of the rigid shaft terminating in a tip configured to puncture the subcutaneous vessel of the patient, and
an enlarged segment, wherein the enlarged segment is positioned a predetermined distance from the tip and has a radius greater than that of the main body; and
a hub connected to the proximal end of the rigid shaft.

10. The needle according to claim 9, wherein the enlarged segment is rounded.

11. The needle according to claim 9, wherein the radius of the enlarged segment is approximately 2-4 mm.

12. The needle according to claim 9, wherein the predetermined distance between the tip and the enlarged segment is greater than a thickness of a wall of the subcutaneous vessel.

13. A method for cannulating a subcutaneous vessel of a patient, comprising:
creating a puncture site in the subcutaneous vessel of the patient;
inserting a cannula assembly through the puncture site and into the subcutaneous vessel of the patient, wherein the cannula assembly comprises
a cannula including a shaft, the shaft defining a distal end of the cannula that is configured to be inserted into the subcutaneous vessel of the patient; and
an elongated sleeve, the elongated sleeve including
a proximal end,
a distal end configured to be inserted into the subcutaneous tissue and positioned proximal to the puncture site of the patient,
a bore extending from the proximal end of the elongated sleeve to the distal end of the elongated sleeve and configured to receive the shaft of the cannula,
a slide mounted for movement with respect to a proximal end of a main body of the elongated sleeve, the slide connected to each wire of a plurality of wires, and
a plurality of channels circumferentially spaced about the bore, the plurality of channels defining a pathway through which at least one of (i) a flow of air can be drawn and (ii) the plurality of wires can be advanced to promote the formation of a seal between the distal end of the elongated sleeve and the subcutaneous vessel around the puncture site, such that, in use, the slide can be selectively moved in a first direction to advance the plurality of wires within the plurality of channels and direct a distal end of the plurality of wires into the subcutaneous vessel of the patient, and such that the slide can be selectively moved in a second direction to retract the plurality of wires within the plurality of channels; and
treating the puncture site, wherein treating the puncture site includes at least one of (iii) drawing the flow of air through the plurality of channels and (iv) drawing the plurality of metal wires through the plurality of channels, subsequent to insertion of the plurality of metal wires into the subcutaneous vessel.

14. The method according to claim 13, wherein the elongated sleeve includes one or more protrusions radially extending from an exterior surface of the elongated sleeve and one or more clips configured to receive a circumferential ridge of the slide, and wherein the one or more clips limit the extent to which the slide can move in the first direction and the one or more protrusions limit the extent to which the slide can move in the second direction.

15. The method according to claim 13, and further comprising:
removing the cannula from the elongated sleeve of the cannula assembly; and
inserting, subsequent to the removal of the cannula from the elongated sleeve, a plug into the bore into the sleeve of the cannula assembly, such that, when the plug is fully inserted into the bore, a distal end of the plug applies a pressure to the puncture site.

16. The method according to claim 15, wherein a thrombotic material configured to promote thrombosis at the puncture site is deposited on the distal end of the plug.

17. The method according to claim 15, wherein at least one electrically conductive plate is connected to the distal end of the plug, and the at least one electrically conductive plate is operably connected to an electrical source, and further comprising:
cauterizing the puncture site with the distal end of the plug.

18. The method according to claim 13, and further comprising:
monitoring for the development of a hematoma around the puncture site by measuring impedance between a first electrode disposed on an outer surface of the distal end of the elongated sleeve and a second electrode disposed on an exterior surface of the skin of the patient.

19. The method according to claim 13, wherein creating the puncture site includes inserting a needle into a wall of the subcutaneous vessel using ultrasound guidance.

20. The method according to claim 19, wherein the needle comprises
a rigid shaft, including
a main body defining a bore for receiving a guidewire, the bore extending from a proximal end of the rigid shaft to a distal end of the rigid shaft, the distal end of the rigid shaft terminating in a tip configured to puncture skin, subcutaneous tissue, and the subcutaneous vessel of the patient, and
an enlarged segment, wherein the enlarged segment is positioned a predetermined distance from the tip and has a radius greater than that of the main body; and
a hub connected to the proximal end of the rigid shaft.
